SEQUENCE LISTING (3 sequences)

SEQ ID NO: 1 - protein, 15 aa, Artificial Sequence
  Artificial Connecting Peptide
GGGGSGGGGSGGGGS

SEQ ID NO: 2 - protein, 109 aa, Homo sapiens
PELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 3 - protein, 106 aa, Homo sapiens [sequence omitted]

(19) United States
(12) Patent Application Publication
(10) Pub. No.: US 2010/0056759 A1
(43) Pub. Date: Mar. 4, 2010

(54) Methods and Compositions for Efficient Removal of Protein A from Binding Molecule Preparations

(75) Inventor: Michael Paglia, Georgetown, MA (US)

Correspondence Address: FISH & RICHARDSON P.C., PO BOX 1022, MINNEAPOLIS, MN, 55440-1022, US

(73) Assignee: TOLERX, INC., Cambridge, MA (US)

(21) Appl. No.: 12/445,839

(22) PCT Filed: Oct. 18, 2007
(86) PCT No.: PCT/US07/22261

§ 371 Date: Apr. 16, 2009

Related U.S. Application Data
(60) Provisional application No. 60/852,728, filed on Oct. 19, 2006.

(51) Int. Cl. C07K 16/00; C07K 1/14

(52) U.S. Cl. 530/387.3; 530/412; 530/388.1

(57) ABSTRACT

The present invention features methods for reducing protein A contamination in a binding molecule preparation, e.g., a therapeutic binding molecule preparation, comprising residual protein A, or fragments thereof.

### RELATED APPLICATIONS

This application claims the benefit of U.S. Provisional Application, 60/852,728, filed Oct. 19, 2006, titled “Methods and Compositions for Efficient Removal of Protein A from Binding Molecule Preparations”, the entire contents of which is incorporated herein by reference.

### BACKGROUND OF THE INVENTION

Monoclonal antibodies are an exciting therapeutic modality useful for indications such as autoimmune disease, infectious disease, cardiovascular disease, transplant rejection, and cancer (Carter, et al. (1992) Proc Natl Acad Sci, USA 89:4285; Anderson, et al. (1996) Biochem Soc Trans 25:705; Baselga, et al. (1996) J Clin Oncol 14:737; Bodley, et al. (1996) Anticancer Res 16:517; Long (1996) Curr Opin Oncol 8:353; Huston (2001) Antibodies 10: 127; and MacConnachie (2000) Crit Care Nurs 16:123). Owing to the large size, complex folding structure, and posttranslational modification of these molecules, e.g., glycosylation, mammalian cell culture is the primary means of expression of these proteins.

Typically, an antibody construct is transfected into a mammalian cell line that is capable of secreting the intact antibody into the surrounding media environment. The growth media that is used is rich in saccharide and amino acid content. The nutrient rich environment that the mammalian cells are cultured in propagates healthy cell growth and antibody secretion. Along with antibody production, however, the mammalian cells also create toxic bi-products via their normal metabolic pathways. The level of metabolic bi-products, such as carbon dioxide and proteases must be tightly controlled in order to ensure that the desired antibody molecules are not damaged in any way and must be removed in a purification step. In addition, any viral particles or other impurities must be removed using purification techniques.

A common step in therapeutic antibody purification is protein A affinity chromatography which selectively binds antibodies in complex mixtures such as harvested cell culture fluid and removes many impurities in a single step. Protein A affinity chromatography consists of bead-like matrices composed of cross-linked polysaccharides, plastic, or glass that are chemically modified allowing for attachment of recombinant protein A via an epoxide linkage. The primary binding site for protein A is the Fc portion of IgG. The antibody containing media is passed over a protein A column under neutral conditions, washed with a series of buffers to remove contaminants and the antibody is eluted from the protein A column under low pH conditions. The nanomolar affinity and IgG specificity of protein A allows for purification of the IgG molecules from the surrounding impurities (including serum, if present) to greater than 90% (Ey, et al. (1978) Immunochemistry 15:429; Surolia, et al. (1982) Trends Biochem 7:74; Lindmark, et al. (1983) J Immunol Meth 62:1; Fuglistaller (1989) J Immunol Meth 124:171; and Reis, et al. (1984) J Clin Lab Immunol 13:75).

The biding sites of the protein A molecule are protease resistant, but upon binding of the IgG antibody, the flexible coil structure of the protein A relaxes making it very susceptible to proteolytic cleavage. Thus, the protein A used in the purification can itself contaminate antibody preparations and leached protein A co-elutes with the product or remains bound to the antibody after elution. In fact, as much as 1.5% of IgG in an antibody preparation can be contaminated with protein A (Gangon, Purification Tools for Monoclonal Antibodies, Validated Biosytems, Tucson, Ariz., 1996). In addition, the ICH S6 guidance (ICH. S6 Preclinical Safety Evaluation of Biotechnology-Derived Pharmaceuticals. Federal Register 62(222), 18 Nov. 1997: 61515) cautions that there are risks of theoretical adverse effects arising from cell substrate derived impurities and other process related impurities which include those derived from cell culture (e.g., antibiotics and media components) and downstream processes (e.g., chemical additives and column leachables) (Simmerman and Donnelly (2005) BioProcess International Vol. 3, No. 6: pp 32-40; F-D-C Reports, The Gold Sheet (2004) Vol. 38, No 9, pp 9-12) owing to the potential for immunogenicity and other adverse physiological responses in humans (Gangon, Purification Tools for Monoclonal Antibodies, Validated Biosystems, Tucson, Ariz., 1996). Therefore, if the mammalian culture conditions result in elevated levels of proteolytic activity, this activity must be suppressed using chelating agents to inhibit the serine protease activity in the culture or the residual protein A must be removed in subsequent purification steps, i.e., polishing steps, such as ion exchange chromatography, size exclusion, or hydrophobic interaction chromatography which are costly and time consuming steps. Furthermore, these methods may not remove protein A to levels acceptable to FDA manufacturing practices. Therefore, there is a need for improved methods to remove contaminating residual protein A from preparations of binding molecules.

### SUMMARY OF THE INVENTION

The present invention is based, at least in part, on the discovery of improved and more efficient methods to purify a binding molecule preparation, e.g., a therapeutic binding molecule preparation. More specifically, it has been discovered that a charge-modified depth filter efficiently removes residual protein A, or fragments thereof, that leach into a binding molecule preparation during the purification process, i.e., during protein A affinity chromatography, by pre-treating the filter with a basic solution, e.g., a solution having a pH of greater than 7. Accordingly, one aspect of the present invention provides methods for reducing protein A contamination in a binding molecule preparation comprising residual protein A, or fragments thereof, from a binding molecule preparation, e.g., a therapeutic binding molecule, preparation the method comprising, contacting a charge-modified depth filter with a solution having a pH greater than about 8 to obtain a pre-treated charge-modified depth filter, contacting the pre-treated charge-modified depth filter with the binding molecule preparation, and collecting the binding molecule preparation that flows through the filter to thereby obtain a binding molecule preparation having reduced protein A contamination.

In one embodiment, the charged-depth filter is an anion exchange filter.

In one embodiment, prior to reducing protein A contamination the binding molecule preparation is purified by a process selected from the group consisting of: centrifugation, depth filtration, protein A affinity chromatography, anion exchange chromatography, cation exchange chromatography, or a combination thereof.

In one embodiment, the binding molecule preparation is further purified by a process selected from the group consisting of: anion exchange chromatography, cation exchange chromatography, filtration, or a combination thereof.

In one embodiment, the charge-modified depth filter comprises a matrix selected from the group consisting of: paper, glass fibers, nylon, polyolefin, carbon, ceramics, diatomaceous Earth and cellulose. In another embodiment, the charge-modified depth filter matrix is cellulose.

In one embodiment, the solution having a pH greater than about 8 is sodium hydroxide. In one embodiment, the concentration of the sodium hydroxide solution is greater than about 0.05 M NaOH.

In one embodiment, the sodium hydroxide is either flushed at a constant flux through the filter or held without flow for greater than 5 minutes and then flushed through the filter.

In one embodiment, the methods of the invention further comprise the step of contacting the pre-treated filter with a sodium phosphate solution or water prior to contact with the binding molecule preparation.

In one embodiment, the initial concentration of residual protein A is greater than 30 ppm. In one embodiment, the concentration of residual protein A is reduced to less than about 20 ppm. In another embodiment, the concentration of residual protein A is reduced to less than about 10 ppm. In yet another embodiment, the concentration of residual protein A is reduced to less than about 5 ppm. In one embodiment, the concentration of residual protein A is reduced to less than about 2.5 ppm. In another embodiment, the concentration of residual protein A is reduced to less than about 1 ppm.

In one embodiment, the binding molecule preparation comprises a monoclonal binding molecule. In another embodiment, the binding molecule preparation comprises a chimeric binding molecule. In one embodiment, the chimeric binding molecule comprises a fusion polypeptide. In yet another embodiment, the binding molecule preparation comprises a humanized binding molecule. In one embodiment, the binding molecule preparation comprises a an IgG1 constant region.

In one embodiment, the binding molecule preparation is obtained from cells cultured in serum-free or serum containing medium.

In one embodiment, the binding molecule is obtained from cells cultured in a hollow fiber or stirred tank bioreactor in batch or fed batch configuration

In one embodiment, the methods of the invention further comprise incorporating the binding molecule preparation into a pharmaceutical formulation.

### DETAILED DESCRIPTION OF THE INVENTION

The present invention is based, at least in part, on the discovery of improved and more efficient methods to purify a binding molecule preparation, e.g., a therapeutic binding molecule preparation. More specifically, it has been discovered that the pretreatment of a charge-modified depth filter with a solution having a basic pH, e.g., greater than about 8, efficiently removes residual protein A, or fragments thereof, from a binding molecule preparation that leach into the preparation during the purification process or remain attached to the binding molecule following the purification process, e.g., following protein A affinity chromatography.

Various aspects of the invention are described in further detail in the following subsections:

### I. DEFINITIONS

As used herein, each of the following terms has the meaning associated with it in this section.

“Protein A” is a polypeptide found naturally in the cell wall of the microorganism Staphylococcus aureus. As used herein “protein A” also refers to protein A made recombinantly. The protein A polypeptide can be divided into structurally and functionally different regions. One region located N-terminally consists of four globular, highly homologous immunoglobulin (IgG) binding units. The second region is a C-terminal region that does not bind IgG and covalently links the polypeptide to the cell wall (Hjelm, et al. (1975) Eur J Biochem 57:395; Sjodahl (1977) Eur J Biochem 73:343). Protein A interacts with IgG via the Fc portion of the heavy chain of IgG of many mammalian species (Forsgren and Sjoquist (1966) J Immunol 97:822; Sjoquist, et al. (1967) Cold Spring Harbor Symp Quant Biol 32:577). Protein A is not specific for IgG since fractions of human IgM IgA, and IgE have also been found to interact (Harboe and Folling (1974) Scand J Immunol 3:471; Johansson and Inganas (1978) Immunol Rev 41:248; Brunda, et al. (1979) J Immunol 123:1457; Field, et al. (1980) J Immunol Methods 32:59).

Protein A is often immobilized and used as the primary capture and purification step in the purification process of binding molecule preparations. The term “residual protein A” refers to protein A, or fragments thereof, which contaminate a binding molecule preparation following protein A affinity chromatography, thus requiring additional downstream purification steps to remove it prior to use, e.g. pharmaceutical formulation.

As used herein “a protein A binding domain” refers to a portion of a polypeptide, i.e., a binding molecule, necessary for binding to a protein A polypeptide, i.e., a protein A binding domain of an Fc portion of an immunoglobulin polypeptide. As used herein “an Fc portion of an immunoglobulin polypeptide”, “Fc fragment”, or “Fc” refers to the dimer of the C-terminal heavy chain constant region of an immunoglobulin molecule. The regions of the heavy chain constant region shown to be necessary for binding to protein A comprise the CH2 and CH3 domains of Fc (Deisenhofer (1981) Biochem 20:2361).

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system, Kabat E A et al. Sequences of Proteins of Immunological Interest. Bethesda, US Department of Health and Human Services, NIH. 1991). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues. An exemplary CH2 sequence is as follows:

PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAK (human IgG1 CH2).

As used herein, the term “CH3 domain” includes the portion of a heavy chain molecule that extends approximately 110 residues from N-terminus of the CH2 domain, e.g., from about residue 341-446, BU numbering system). The CH3 domain typically forms the C-terminal portion of the antibody. In some immunoglobulins, however, additional domains may extend from CH3 domain to form the C-terminal portion of the molecule (e.g. the CH4 domain in the μ chain of IgM and the ε chain of IgE). An exemplary CH3 sequence is as follows:

QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK (human IgG1 CH3).

The term “binding molecule” as used herein comprises polypeptides, natural or recombinant, that bind to protein A, i.e., that contain at least one protein A binding domain.

In one embodiment a binding molecule is an “antibody”, e.g., naturally occurring or genetically engineered antibody molecules. As used herein “antibody” refers to a polypeptide having a combination of two heavy and two light chains which have significant known specific immunoreactive activity to an antigen of interest. As used herein, the term “immunoglobulin” includes a polypeptide having a combination of two heavy and two light chains whether or not it possesses any relevant specific immunoreactivity.

Antibodies and immunoglobulins comprise light and heavy chains, with or without an interchain covalent linkage between them. The basic immunoglobulin structures in vertebrate systems are well understood in the art.

The generic term “immunoglobulin” comprises five distinct classes of antibody that can be distinguished biochemically. All five classes of antibodies are within the scope of the present invention, the following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

In one embodiment, the polypeptides of the invention are modified antibodies. As used herein, the term “modified antibody” includes synthetic forms of antibodies which are altered such that they are not naturally occurring. Such modifications may include, but are not limited to, the removal of N-linked or O-linked naturally occurring glycosylation sites, mutation of the regions responsible for FCγRII binding, and any other naturally occurring posttranslational modifications. Bolt et. al (1993) Eur. J. Immunol. 23: 403-411; Woodle et. al (1998) Translation Proceedings, 30: 1369-1370; Xu et. al. (2000) Cellular Immunology, 200: 16-26. Preferably, such modified antibodies retain the ability to bind protein A.

The term antibody, as used herein, includes full length antibodies, monoclonal antibodies (including full length monoclonal antibodies), polyclonal antibodies, multispecific antibodies (e.g., bispecific antibodies), human, humanized or chimeric antibodies, and antibody fragments that comprise a protein A binding domain, engineered forms of antibodies, e.g., scFv molecules, diabodies, linear antibodies, single-chain antibody molecules; and multispecific antibodies formed from antibody fragments, so long as they exhibit the desired activity.

In one embodiment, a binding molecule of the invention comprises an antibody binding site. For example, in one embodiment, a binding molecule of the invention is a full-length antibody molecule. In another embodiment, a binding molecule of the invention is a fragment of an antibody molecule. In another embodiment, binding molecule of the invention is a modified or synthetic antibody molecule.

The binding molecules of the invention may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule, so long as it contains at least one protein A binding domain.

An “antigen” is an entity (e.g., a proteinaceous entity or peptide) to which an antibody specifically binds.

The term “monoclonal binding molecule”, e.g., a monoclonal antibody, as used herein refers to a binding molecule obtained from a population of substantially homogeneous binding molecules. Monoclonal binding molecules are highly specific, being directed against a single antigenic site. Furthermore, in contrast to polyclonal binding molecule, e.g., polyclonal antibody, preparations which typically include different binding molecules directed against different determinants (epitopes), each monoclonal binding molecule is directed against a single determinant on an antigen. The modifier “monoclonal” indicates the character of the binding molecule as being obtained from a substantially homogeneous population of binding molecules, and is not to be construed as requiring production of the binding molecule by any particular method. For example, the monoclonal binding molecules to be used in accordance with the present invention may be made by the hybridoma method first described by Kohler, et al., Nature 256:495 (1975), or may be made by recombinant DNA methods (see, e.g., U.S. Pat. No. 4,816,567). The “monoclonal binding molecules” may also be isolated from phage antibody libraries using the techniques described in Clackson, et al., Nature 352:624-628 (1991) and Marks et al., J. Mol Biol. 222:581-597 (1991), for example (discussed further below).

A “chimeric” binding molecule, as used herein, refers to a polypeptide that comprises a first amino acid sequence linked to a second amino acid sequence with which it is not naturally linked in nature, e.g., a protein A binding domain polypeptide linked to a protein of interest. A chimeric protein may be created, for example, by chemical synthesis, or by creating and translating a polynucleotide in which the peptide regions are encoded in the desired relationship (e.g., genetic engineering). The term “chimeric binding molecule”, as used herein, also refers to a binding molecule comprising amino acid sequences derived from different species. Exemplary chimeric binding molecules include, for example, chimeric antibodies and fusion proteins.

In one embodiment, a binding polypeptide of the invention is a chimeric fusion proteins. In one embodiment, the chimeric binding molecule is a chimeric antibody.

The monoclonal binding molecules herein specifically include “chimeric” fusion proteins in which a portion of the heavy and/or light chain is identical with or homologous to corresponding sequences in binding molecules derived from a particular species or belonging to a particular antibody class or subclass, while the remainder of the chain(s) is identical with or homologous to corresponding sequences in binding molecules derived from another species or belonging to another antibody class or subclass, as well as fragments of such binding molecules, so long as they exhibit the desired biological activity (U.S. Pat. No. 4,816,567; and Morrison, et al., Proc. Natl. Acad Sci. USA 81:6851-6855 (1984)). “Humanized” forms of non-human (e.g., murine) binding molecules may also be used in the methods of the invention. These are chimeric antibodies which contain minimal sequence derived from non-human binding molecule. For the most part, humanized binding molecules are human binding molecules (acceptor/recipient binding molecule) in which residues from a hyper-variable region are replaced by residues from a hypervariable region of a non-human species (donor binding molecule) such as mouse, rat, rabbit or nonhuman primate having the desired specificity, affinity, and capacity. In some instances, Fv framework region (FR) residues of the human binding molecule are altered, e.g., replaced by, substituted, or backmutated to corresponding non-human residues. Furthermore, humanized binding molecules may comprise residues which are not found in the recipient binding molecule or in the donor binding molecule. These modifications are generally made to further refine binding molecule performance. In general, the humanized binding molecule will comprise substantially all of at least one, and typically two, variable domains, in which all or substantially all of the hypervariable loops correspond to those of a non-human binding molecule and all or substantially all of the FR regions are those of a human binding molecule sequence. The humanized binding molecule optionally also will comprise at least a portion of a binding molecule constant region (Fc), typically that of a human binding molecule. For further details, see Jones, et al., Nature 321:522-525 (1986); Riechmann, et al., Nature 332:323-329 (1988); and Presta, Curr. Op. Struct. Biol. 2:593-596 (1992).

The term “diabodies” refers to small antibody fragments with two antigen-binding sites, which fragments comprise a heavy-chain variable domain (VH) connected to a light-chain variable domain (VL) on the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Holliger et al., Proc. Natl. Acad. Sci. USA, 90:6444 6448 (1993).

The term “engineered” or “recombinant” binding molecule, as used herein includes binding molecules that are prepared, expressed, created or isolated by recombinant means, such as binding molecules expressed using a recombinant expression vector transfected into a host cell, binding molecules isolated from a recombinant, combinatorial binding molecule library, binding molecules isolated from an animal (e.g., a mouse) that is transgenic for human immunoglobulin genes (see e.g., Taylor, L. D., et al., (1992) Nucl. Acids Res. 20:6287-6295) or binding molecules prepared, expressed, created or isolated by any other means that involves splicing of human binding molecule gene sequences to other DNA sequences. In certain embodiments, however, such recombinant human binding molecules are subjected to in vitro mutagenesis (or, when an animal transgenic for human Ig sequences is used, in vivo somatic mutagenesis) and thus the amino acid sequences of the VH and VL regions of the recombinant binding molecules are sequences that, while derived from and related to human germline VH and VL sequences, may not naturally exist within the human binding molecule germline repertoire in vivo.

In another embodiment a binding molecule of the invention comprises an amino acid sequence not derived from an antibody molecule linked to a protein A binding domain or a polypeptide comprising a protein A binding domain. In another embodiment a binding molecule of the invention comprises at least one amino acid sequence derived from an antibody molecule linked to a protein A binding domain or a polypeptide comprising a protein A binding domain. In one embodiment, the chimeric binding molecule is a fusion protein.

The fusion proteins useful in the methods of the present invention comprise a protein A binding domain or a polypeptide comprising a protein A binding domain linked to a polypeptide of interest. For example, a fusion protein of the invention may comprise a protein of interest fused to a protein A binding domain, e.g., an Fc polypeptide, such that the resulting Fc-fusion protein is secreted into the culture medium.

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORE, in a manner that maintains the correct reading frame of the original ORFs. Thus, the resulting recombinant fusion protein is a single protein containing two or more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

An “isolated binding molecule”, as used herein, refers to a binding molecule that is substantially free of other binding molecules having different antigenic specificities. Moreover, an isolated binding molecule may be substantially free of other cellular material and/or chemicals. An “isolated” binding molecule is one which has been identified and separated and/or recovered from a component of its natural environment. Contaminant components of its natural environment include, e.g. materials which would interfere with diagnostic or therapeutic uses for the binding molecule, and may include enzymes, hormones, and other proteinaceous or nonproteinaceous solutes.

The term “recombinant host cell” (or simply “host cell”), as used herein, refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “depth filter” (also referred to as “prefilter” or “media filter”) as used herein, refers to a filter comprising a porous medium that is capable of retaining particles from the mobile liquid phase throughout the filter rather than just on the surface of the filter.

Depth filters are often used to remove larger, insoluble contaminants, e.g., microorganisms, cellular debris, nucleic acids, host-cell-derived aggregates prior to further downstream purification steps, e.g., chromatography or micromembrane filtration steps. Depth filters are often composed of a fibrous bed of, for example, cellulose or polypropylene fibers, along with a filter aid or “matrix”, e.g., paper, glass fibers, nylon, polyolefin, carbon, ceramics, diatomaceous earth, or cellulose, which provides a high surface area to the filter. In one embodiment, the charge-modified depth filter comprises a matrix selected from the group consisting of paper, glass fibers, nylon, polyolefin, carbon, ceramics, diatomaceous earth, and cellulose. In another embodiment, the charge-modified depth filter comprises a matrix of cellulose. A binder is also added to create flat sheets of filter medium. Some depth filters can be imparted with a charge, i.e., a positive (cationic) or negative (anionic) charge, and are referred to herein as “charge-modified depth filters”. In one embodiment of the invention, a charge-modified depth filter is an anion exchange filter. A charge-modified depth filter typically has two distinct zones and, thus, two distinct mechanisms for the removal of such contaminants. The first is a physical means of capture of contaminant where particles become entrapped within a matrix. The second means is electrokinetic absorption which removes, e.g., bacteria, fungi viruses, and other negatively charged contaminants, that are smaller than the physical pore size of the media making up the second means. The two or more zones or layers may be composed of similar materials (in which the materials are formulated and processed such that they have different retention capabilities), or may be composed of different materials having distinctly different particle retention characteristics. The two or more zones may be contiguous or non-contiguous with one another as long as the fluid being filtered communicates between the zones.

Charge-Depth filters are manufactured by numerous companies, such as, for example, Cuno, Inc., ErtelAlsop, Filtrox AG, GE Infrastructure Water and Process Technologies, Meissner Filtration Products, Inc., Millipore Corp., Pall, Corp., Sartorius AG, and US Filter. Several product lines of charge-depth filters are available from all of these companies. Non-limiting examples of such charge-depth filters include, ZetaPlus, PolyNET, Betafine, Disk-Pak, Accusclae, PharmaScale, PuraFix.

As used herein, “chromatography” is any method of separating analytes, e.g., amino acid molecules (proteins and polypeptides), nucleic acid molecules, fatty acids, steroids, carbohydrates, lipids, etc., within a given sample such that the native state of the given analyte is retained. Separation of one analyte from other analytes within a given sample for the purpose of enrichment, purification and/or analysis, may be achieved by methods including, but not limited to, affinity chromatography, size exclusion chromatography, ion exchange chromatography, hydrophobic and hydrophilic interaction chromatography, metal affinity chromatography, or ligand affinity chromatography wherein “ligand” refers to proteins, e.g., protein A, antibodies, or DNA. Generally, chromatography uses biologically active surfaces as adsorbents to selectively accumulate certain analytes. The absorbent may be in the form of a resin or a membrane.

“Affinity chromatography” is a method of separating analytes based on the recognition of an analyte by a substance, such as that between antigen, e.g., protein A, and binding molecule, enzyme and substrate, or receptor and ligand. Affinity chromatography combines the size fractionation capability of gel permeation chromatography with the ability to design a stationary phase comprising an absorbent that reversibly binds to a known subset of molecules. The absorbent may be synthesized by covalently coupling a ligand, e.g., protein A, for example to an insoluble matrix. The column material is then allowed to adsorb the desired analyte from solution. Subsequently, the conditions are changed to those under which binding does not occur and the analyte is eluted.

“Ion exchange chromatography” is a method of separating analytes based on the charge properties of the molecules. The absorbent used in ion exchange chromatography displays ionic functional groups that interact with analyte ions of opposite charge. Therefore, during ion exchange chromatography, analytes are retained based on ionic interactions with the absorbent. In one embodiment, ion exchange chromatography is “anionic exchange chromatography”. Anion exchange chromatography retains negatively charged anions using positively charged functional group on the absorbent, e.g., a quaternary ammonium cation. In another embodiment, ion exchange chromatography is “cationic exchange chromatography”. Cation exchange chromatography retains positively charged cations using negatively charged functional group on the absorbent, e.g., a phosphonic acid anion.

The term “adsorbent” (also referred to a “capture reagents” or “affinity reagents”) refers to any material that is capable of accumulating (binding) an analyte. The adsorbent typically coats a biologically active surface and is composed of a single material or a plurality of different materials that are capable of binding an analyte. Such materials include, but are not limited to, anion exchange materials, cation exchange materials, metal chelators, polynucleotides, oligonucleotides, peptides, antibodies, metal chelators, etc.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

Various aspects of the invention are described in further detail in the following subsections.

### II. Binding Molecules for Use in the Purification Methods of the Invention

A. Antibodies or Portions Thereof

In one embodiment, a binding molecule of the invention is an antibody molecule or modified antibody molecule. In another embodiment, a binding molecule of the invention comprises an antibody binding site and a protein A binding domain.

Antibodies may be made using art recognized protocols. For example, antibodies may be raised in mammals by multiple subcutaneous or intraperitoneal injections of the relevant antigen and, optionally, an adjuvant.

Rabbits or guinea pigs are typically used for making polyclonal binding molecules. Exemplary preparation of polyclonal binding molecules, e.g., for passive protection, can be performed as follows. Animals are immunized with 100 μg antigen, plus adjuvant, and euthanized at 4-5 months. Blood is collected and IgG is separated from other blood components. Binding molecules specific for the immunogen may be partially purified by affinity chromatography. An average of about 0.5-1.0 mg of immunogen-specific binding molecule is obtained per animal, giving a total of 60-120 mg.

Optionally, the immunogen can be administered with an adjuvant. The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages. Several types of adjuvants can be used as described below. Complete Freund's adjuvant followed by incomplete adjuvant is preferred for immunization of laboratory animals.

The production of non-human monoclonal binding molecules, e.g., murine, guinea pig, primate, rabbit or rat, can be accomplished by, for example, immunizing the animal with an antigen of interest or with a nucleic acid molecule encoding the antigen of interest. For example, a binding molecule may be made by placing the gene encoding an antigen of interest in an expression vector and immunizing animals by multiple subcutaneous or intraperitoneal injections of the relevant antigen (e.g., purified tumor associated antigens or cells or cellular extracts comprising such antigens) and, optionally, an adjuvant. A longer polypeptide comprising the antigen of interest or an immunogenic fragment of the antigen of interest or anti-idiotypic binding molecule of the antigen of interest can also be used. (see, for example, Harlow & Lane, supra, incorporated by reference for all purposes). Such an immunogen can be obtained from a natural source, by peptide synthesis or by recombinant expression. Optionally, the immunogen can be administered, fused or otherwise complexed with a carrier protein, as described below. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature, 256:495 (1975)) the relatively short-lived, or mortal, lymphocytes from a mammal which has been injected with antigen are fused with an immortal tumor cell line (e.g. a myeloma cell line), thus, producing hybrid cells or “hybridornas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

In another embodiment, DNA encoding a binding molecule may be readily isolated and sequenced using conventional procedures (e.g., by using oligonucleotide probes that are capable of binding specifically to genes encoding the heavy and light chains of murine binding molecules). The isolated and subcloned hybridoma cells serve as a preferred source of such DNA Once isolated, the DNA may be placed into expression vectors, which are then transfected into prokaryotic or eukaryotic host cells such as E. coli cells, simian COS cells, Chinese Hamster Ovary (CHO) cells, or myeloma cells that do not otherwise produce immunoglobulins. More particularly, the isolated DNA (which may be synthetic as described herein) may be used to clone constant and variable region sequences for the manufacture of binding molecules as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. Transformed cells expressing the desired antibody may be produced in relatively large quantities to provide clinical and commercial supplies of the binding molecule.

Those skilled in the art will also appreciate that DNA encoding binding molecules or fragments thereof (e.g., antigen binding sites) may also be derived from antibody phage libraries, e.g., using pd phage or Fd phagemid technology. Exemplary methods are set forth, for example, in EP 368 684 B1; U.S. Pat. No. 5,969,108, Hoogenboom, H. R. and Chames. 2000. Immunol Today 21:371; Nagy et al., 2002. Nat. Med. 8:801; Huie et al., 2001. Proc. Natl. Acad. Sci. USA 98:2682; Lui et al., 2002. J. Mol. Biol. 315:1063, each of which is incorporated herein by reference. Several publications (e.g., Marks et al., Bio/Technology 10:779-783 (1992)) have described the production of high affinity human binding molecules by chain shuffling, as well as combinatorial infection and in vivo recombination as a strategy for constructing large phage libraries. In another embodiment, Ribosomal display can be used to replace bacteriophage as the display platform (see, e.g., Hanes et al., 2000. Nat. Biotechnol. 18:1287; Wilson et al., 2001. Proc. Natl. Acad. Sci. USA 98:3750; or Irving et al., 2001 J. Immunol. Methods 248:31. In yet another embodiment, cell surface libraries can be screened for binding molecules (Boder et al., 2000. Proc. Nat. Acad. Sci. USA 97:10701; Daugherty et al., 2000 J. Immunol. Methods 243:211. Such procedures provide alternatives to traditional hybridoma techniques for the isolation and subsequent cloning of monoclonal binding molecules.

Yet other embodiments of the present invention comprise human or substantially human binding molecules generated in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human binding molecules upon antigen challenge. Another means of generating human binding molecules using SCID mice is disclosed in U.S. Pat. No. 5,811,524 which is incorporated herein by reference. It will be appreciated that the genetic material associated with these human binding molecules may also be isolated and manipulated as described herein.

Alternatively, binding molecule-coding sequences can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

Recombinant binding molecules may also be generated by the methods disclosed in Newman, Biotechnology, 10: 1455-1460 (1992). Specifically, this technique results in the generation of primatized binding molecules that contain monkey variable domains and human constant sequences. This reference is incorporated by reference in its entirety herein. Moreover, this technique is also described in U.S. Pat. Nos. 5,658,570, 5,693,780, and 5,756,096, each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Moreover, genetic sequences useful for producing polypeptides encoding a binding molecule for use in the methods of the present invention may be obtained from a number of different sources. For example, as discussed above, a variety of human antibody genes are available in the form of publicly accessible deposits. Many sequences of antibodies and antibody-encoding genes have been published and suitable antibody genes can be chemically synthesized from these sequences using art recognized techniques. Oligonucleotide synthesis techniques compatible with this aspect of the invention are well known to the skilled artisan and may be carried out using any of several commercially available automated synthesizers. In addition, DNA sequences encoding several types of heavy and light chains set forth herein can be obtained through the services of commercial DNA synthesis vendors. The genetic material obtained using any of the foregoing methods may then be altered or synthesized.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in one of skill in the art.

In one embodiment, cDNAs that encode the light and the heavy chains of a binding molecule may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the binding molecule light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

In one embodiment, a binding molecule of the invention comprises or consists of an antigen binding fragment of an antibody. The term “antigen-binding fragment” refers to a polypeptide fragment of an immunoglobulin or antibody that binds antigen or competes with intact antibody (i.e., with the intact antibody from which they were derived) for antigen binding (i.e., specific binding). As used herein, the term “fragment” of an antibody molecule includes antigen-binding fragments of antibodies, for example, an antibody light chain (VL), an antibody heavy chain (VH, a single chain antibody (scFv), a F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means.

In one embodiment, a binding molecule for use in the methods of the invention is an engineered or modified antibody. Engineered forms of antibodies include, for example, minibodies, diabodies, diabodies fused to CH3 molecules, tetravalent antibodies, intradiabodies (e.g., Jendreyko et al., 2003. J. Biol. Chem. 278:47813), bispecific antibodies, fusion proteins (e.g., antibody cytokine fusion proteins) or, bispecific antibodies. Other immunoglobulins (Ig) and certain variants thereof are described, for example in U.S. Pat. No. 4,745,055; EP 256,654; Faulkner et al., Nature 298:286 (1982); EP 120,694; EP 125,023; Morrison, J. Immun. 123:793 (1979); Kohler et al., Proc. Natl. Acad. Sci. USA 77:2197 (1980); Raso et al., Cancer Res. 41:2073 (1981); Morrison et al., Ann. Rev. Immunol. 2:239 (1984); Morrison, Science 229:1202 (1985); Morrison et al., Proc. Natl. Acad. Sci. USA 81:6851 (1984); EP 255,694; EP 266,663; and WO 88/03559. Reassorted immunoglobulin chains also are known. See, for example, U.S. Pat. No. 4,444,878; WO 88/03565; and EP 68,763 and references cited therein.

In one embodiment, the modified antibodies of the invention are minibodies. Minibodies are dimeric molecules made up of two polypeptide chains each comprising an ScFv molecule (a single polypeptide comprising one or more antigen binding sites, e.g., a VL domain linked by a flexible linker to a VH domain fused to a CH3 domain via a connecting peptide.

ScFv molecules can be constructed in a VH-linker-VL orientation or VL-linker-VH orientation.

The flexible hinge that links the VL and VH domains that make up the antigen binding site preferably comprises from about 10 to about 50 amino acid residues. An exemplary connecting peptide for this purpose is (Gly4Ser)3 (Huston et al., 1988. Proc. Natl. Acad. Sci. USA 85:5879). Other connecting peptides are known in the art.

Methods of making single chain antibodies are well known in the art, e.g. Ho et al., 1989. Gene 77:51; Bird et al., 1988 Science 242:423; Pantoliano et al., 1991. Biochemistry 30:10117; Milenic et al., 1991. Cancer Research 51:6363; Takkinen et al., 1991. Protein Engineering 4:837.

Minibodies can be made by constructing an ScFv component and connecting peptide-CH3 component using methods described in the art (see, e.g., U.S. Pat. No. 5,837,821 or WO 94/09817A1). These components can be isolated from separate plasmids as restriction fragments and then ligated and recloned into an appropriate vector. Appropriate assembly can be verified by restriction digestion and DNA sequence analysis.

Diabodies are similar to scFv molecules, but usually have a short (less than 10 and preferably 1-5) amino acid residue linker connecting both V-domains, such that the VL and VH domains on the same polypeptide chain can not interact Instead, the VL and VH domain of one polypeptide chain interact with the VH and VL domain (respectively) on a second polypeptide chain (WO 02/02781). In one embodiment, a binding molecule for use in the methods of the invention is a diabody fused to at least one heavy chain portion. In one embodiment, a binding molecule for use in the methods of the invention is a diabody fused to a CH3 domain.

Other forms of modified antibodies are also within the scope of the instant invention (e.g., WO 02/02781 A1; 5,959,083; 6,476,198 B1; US 2002/0103345 A1; WO 00/06605; Byrn et al., 1990. Nature. 344:667-70; Chamow and Ashkenazi. 1996. Trends Biotechnol. 14:52).

In one embodiment, a binding molecule for use in the methods of the invention can be derivatized or linked to another functional molecule (e.g., another peptide or protein). Accordingly, a binding molecule for use in the methods of the invention may be derivatized and otherwise modified. For example, a binding molecule of the invention can be functionally linked (by chemical coupling, genetic fusion, noncovalent association or otherwise) to one or more other molecular entities, such as another binding molecule (e.g., a bispecific antibody or a diabody), a detectable agent, a cytotoxic agent, a pharmaceutical agent, and/or a protein or peptide that can mediate association of the binding molecule with another molecule (such as a streptavidin core region or a polyhistidine tag).

One type of derivatized binding molecule is produced by crosslinking two or more binding molecules (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which a binding molecule of the invention may be derivatized include fluorescent compounds. Exemplary fluorescent detectable agents include fluorescein, fluorescein isothiocyanate, rhodamine, 5-dimethylamine-1-naphthalenesulfonyl chloride, phycoerythrin and the like. A binding molecule may also be derivatized with detectable enzymes, such as alkaline phosphatase, horseradish peroxidase, glucose oxidase and the like. When a binding molecule is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. A binding molecule may also be derivatized with biotin, and detected through indirect measurement of avidin or streptavidin binding.

B. Fusion Proteins

In one embodiment, a binding molecule is a fusion protein. The invention also pertains to binding molecules which comprise one or more protein A binding domains fused to a non-antibody amino acid sequence. The fusion proteins of the invention comprise a protein A binding domain operatively linked to an amino acid sequence which is biologically active. For example, a biologically active amino acid sequence includes a binding sequence such as the receptor binding portion of a ligand molecule or the ligand binding portion of a receptor molecule. The subject fusion proteins may be bispecific (with one binding site for a first target and a second binding site for a second target) or may be multivalent (with two binding sites for the same target).

Within the fusion protein, the term “operatively linked” is intended to indicate that the protein A binding domain polypeptide and the other polypeptide are fused in-frame to each other. The other polypeptide may be fused to the N-terminus or C-terminus of the protein A binding domain polypeptide.

In one embodiment, the sequence of the ligand or receptor domain is fused to the N-terminus of the Fc domain of an immunoglobulin molecule. It is also possible to fuse the entire heavy chain constant region to the sequence of the ligand or receptor domain. In one embodiment, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. The precise site at which the fusion is made is not critical; particular sites are well known and may be selected in order to optimize the biological activity, secretion, or binding characteristics of the molecule. Methods for making fusion proteins are known in the art.

For bispecific fusion proteins, the fusion proteins may be assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Additional exemplary ligands and their receptors that may be included in the subject binding molecules include the following molecules or molecules which bind to them:

Cytokines and Cytokine Receptors

Cytokines have pleiotropic effects on the proliferation, differentiation, and functional activation of lymphocytes. Various cytokines, or receptor binding portions thereof, can be utilized in the fusion proteins of the invention. Exemplary cytokines include the interleukins (e.g. IL-1, IL-2, IL-3, 14, IL-5, IL-6, IL-7, IL-S, IL-10, IL-11, IL-12, IL-13, and IL-18), the colony stimulating factors (CSFs) (e.g. granulocyte CSF (G-CSF), granulocyte-macrophage CSF (GM-CSF), and monocyte macrophage CSF (M-CSF)), tumor necrosis factor (TNF) alpha and beta, and interferons such as interferon-α, β, or γ (U.S. Pat. Nos. 4,925,793 and 4,929,554).

Cytokine receptors typically consist of a ligand-specific alpha chain and a common beta chain. Exemplary cytokine receptors include those for GM-CSF, IL-3 (U.S. Pat. No. 5,639,605), IL-4 (U.S. Pat. No. 5,599,905), IL-5 (U.S. Pat. No. 5,453,491), IFNγ (EP0240975), and the TNF family of receptors (e.g., TNFα (e.g. TNFR-1 (EP 417, 563), TNFR-2 (EP 417,014) lymphotoxin beta receptor).

Adhesion Proteins

Adhesion molecules are membrane-bound proteins that allow cells to interact with one another. Various adhesion proteins, including leukocyte horning receptors and cellular adhesion molecules, of receptor binding portions thereof, can be incorporated in a binding molecule of the invention. Leucocyte homing receptors are expressed on leucocyte cell surfaces during inflammation and include the β-1 integrins (e.g. VLA-1, 2, 3, 4, 5, and 6) which mediate binding to extracellular matrix components, and the β2-integrins (e.g. LFA-1, LPAM-1, CR3, and CR4) which bind cellular adhesion molecules (CAMs) on vascular endothelium. Exemplary CAMs include ICAM-1, ICAM-2, VCAM-1, and MAdCAM-1. Other CAMs include those of the selectin family including E-selectin, L-selectin, and P-selectin.

Chemokines

Chemokines, chemotactic proteins which stimulate the migration of leucocytes towards a site of infection, can also be incorporated into a binding molecule of the invention. Exemplary chemokines include Macrophage inflammatory proteins (MIP-1-α and MIP-1-β), neutrophil chemotactic factor, and RANTES (regulated on activation normally T-cell expressed and secreted).

Growth Factors and Growth Factor Receptors

Growth factors or their receptors (or receptor binding or ligand binding portions thereof) or molecules which bind to them may be incorporated in the binding molecule of the invention. Exemplary growth factors include angiopoietin, Vascular Endothelial Growth Factor (VEGF) and its isoforms (U.S. Pat. No. 5,194,596); Epidermal Growth Factors (EGFs); Fibroblastic Growth Factors (FGF), including aFGF and bFGF; atrial natriuretic factor (ANF); hepatic growth factors (HGFs; U.S. Pat. Nos. 5,227,158 and 6,099,841), neurotrophic factors such as bone-derived neurotrophic factor (BDNF), neurotrophin-3, -4, -5, or -6 (NT-3, NT4, NT-5, or NT-6), or a nerve growth factor such as NGF-β platelet-derived growth factor (PDGF) (U.S. Pat. Nos. 4,889,919, 4,845,075, 5,910,574, and 5,877,016); transforming growth factors (TGF) such as TGF-alpha and TGF-beta (WO 90/14359), osteoinductive factors including bone morphogenetic protein (BMP); insulin-like growth factors-I and -II (IGF-I and IGF-II; U.S. Pat. Nos. 6,403,764 and 6,506,874); Erythropoietin (EPO); stem-cell factor (SCF), thrombopoietin (c-Mpl ligand), and the Wnt polypeptides (U.S. Pat. No. 6,159,462).

Exemplary growth factor receptors which may be used include EGF receptors (EGFRs); VEGF receptors (e.g. Flt1 or Flk1/KDR), PDGF receptors (WO 90/14425); HGF receptors (U.S. Pat. Nos. 5,648,273, and 5,686,292); IGF receptors (e.g. IGFR1 and IGFR2) and neurotrophic receptors including the low affinity receptor (LNGFR), also termed as p75^{NTR} or p75, which binds NGF, BDNF, and NT-3, and high affinity receptors that are members of the trk family of the receptor tyrosine kinases (e.g. trkA, trkB (EP 455,460), trkC (EP 522,530)). In another embodiment, both IGFR1 and VEGF are targeted. In yet another embodiment, VLA4 and VEGF are targeted.

Other cell surface receptors and/or their ligands can also be targeted (e.g., the TNF family receptors or their ligands (as described in more detail herein).

Hormones

Exemplary growth hormones or molecules which bind to them for use as targeting agents in the binding molecule of the invention include renin, human growth hormone (HGH; U.S. Pat. No. 5,834,598), N-methionyl human growth hormone; bovine growth hormone; growth hormone releasing factor; parathyroid hormone (PTH); thyroid stimulating hormone (TSH); thyroxine; proinsulin and insulin (U.S. Pat. Nos. 5,157,021 and 6,576,608); follicle stimulating hormone (FSH), calcitonin, luteinizing hormone (LH), leptin, glucagons; bombesin; somatropin; mullerian-inhibiting substance; relaxin and prorelaxin; gonadotropin-associated peptide; prolactin; placental lactogen; OB protein; or mullerian-inhibiting substance.

Clotting Factors

Exemplary blood coagulation factors for use as targeting agents in the binding molecules of the invention include the clotting factors (e.g., factors V, VII, VIII, X, IX, XI, XII and XIII, von Willebrand factor); tissue factor (U.S. Pat. Nos. 5,346,991, 5,349,991, 5,726,147, and 6,596,840); thrombin and prothrombin; fibrin and fibrinogen; plasmin and plasminogen; plasminogen activators, such as urokinase or human urine or tissue-type plasminogen activator (t-PA).

Fusion proteins are taught, e.g., in WO0069913A1 and WO0040615A2. Fusion proteins can be prepared using methods that are well known in the art (see for example U.S. Pat. Nos. 5,116,964 and 5,225,538). Ordinarily, the ligand or receptor domain is fused C-terminally to the N-terminus of the constant region of the heavy chain (or heavy chain portion) and in place of the variable region. Any transmembrane regions or lipid or phospholipids anchor recognition sequences of ligand binding receptor are preferably inactivated or deleted prior to fusion. DNA encoding the ligand or receptor domain is cleaved by a restriction enzyme at or proximal to the 5′ and 3′ ends of the DNA encoding the desired ORF segment. The resultant DNA fragment is then readily inserted into DNA encoding a heavy chain constant region. The precise site at which the fusion is made may be selected empirically to optimize the secretion or binding characteristics of the soluble fusion protein.

Preferably, a fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a protein A binding domain polypeptide (e.g., InVivoGen). A polypeptide of interest-encoding nucleic acid can be cloned into such an expression vector such that the fusion moiety is linked in-frame to the protein A binding domain protein. Subsequently, the DNA encoding the fusion protein is transfected into a host cell for expression.

### III. Preparation of Binding Molecules

While a binding molecule preparation may be produced by many methods known to one of skill in the art and described herein (e.g. peptic cleavage of intact binding molecules), it is preferably made recombinantly. Various techniques for production of binding molecules, including binding molecule fragments, are described below.

Furthermore, since the methods of the invention are not based on the particular specificity of a binding molecule, e.g., antigen specificity, or a particular polypeptide sequence, the methods of the invention may be used to reduce protein A contamination in a binding molecule preparation having any specificity or sequence of interest (so long as the binding molecule comprises at least one protein A binding domain). Nevertheless, in one embodiment, the binding molecule for use in the methods of the invention is an anti-CD3 binding molecule. In one embodiment the anti-CD3 binding molecule is a monoclonal binding molecule. In one embodiment the anti-CD3 binding molecule is a humanized binding molecule. In one embodiment, the humanized binding molecule comprises murine or hamster CDRs and a human IgG1 heavy chain. In another embodiment, the anti-CD3 binding molecule is a chimeric binding molecule. In another embodiment, the anti-CD3 binding molecule has a reduced effector (i.e. lytic) function. In one embodiment, the anti-CD3 binding molecule with reduced effector function comprises an Fc portion that is aglycosylated. In one embodiment, the anti-CD3 binding molecule with reduced effector function comprises alanines substituted for leucines at positions 234 and 235 (Woodle E S, et al. (1998) Transplant Proc. 30:1369-1370). In one embodiment, the anti-CD3 binding molecule is TRX4 (see, for example U.S. Pat. Nos. 5,968,509; 5, 585,097; and 6,706,265; Bolt, S., et al., (1993) Eur. J. Immunol. 23, 403-411), HUM291 (Cole, M. S., et al., (1999) Transplantation 68(4):563-71), and Visilizumab (Carpenter, P. A., et al., (2002) Blood 99(8):2712-9).

Following manipulation of the isolated genetic material to provide polypeptides of the invention as set forth above, the genes are typically inserted in an expression vector for introduction into host cells that may be used to produce the desired quantity of polypeptide that, in turn, provides the binding molecules for use in the methods of the invention.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In addition to the binding molecule genes, the recombinant expression vectors of the invention carry regulatory sequences that control the expression of the binding molecule chain genes in a host cell. The term “regulatory sequence” includes promoters, enhancers and other expression control elements (e.g., polyadenylation signals) that control the transcription or translation of the binding molecule chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP) and polyoma. For further description of viral regulatory elements, and sequences thereof, see e.g., U.S. Pat. No. 5,168,062 by Stinski U.S. Pat. No. 4,510,245 by Bell et al., and U.S. Pat. No. 4,968,615 by Schaffner, et al.,

In addition to the binding molecule genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Commonly, expression vectors also contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

Once the vector or DNA sequence encoding a binding molecule has been prepared, the expression vector(s) may be introduced into an appropriate host cell using standard techniques. That is, the host cells may be transformed. Introduction of the plasmid into the host cell can be accomplished by various techniques well known to those of skill in the art. These include, but are not limited to, transfection (including electrophoresis and electroporation), protoplast fusion, lipofection, biolistics, calcium phosphate precipitation, DEAE-dextran transfection, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988; Sambrook et al., Molecular Cloning: A Laboratory Manual (Cold Spring Harbor Press, 2nd ed., 1989) (incorporated by reference in its entirety for all purposes). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

The transformed cells are grown under conditions appropriate to the production of binding molecule, and assayed for binding molecule protein synthesis. It is possible to express a binding molecule of the invention in either prokaryotic or eukaryotic host cells.

As used herein, the term “transformation” shall be used in a broad sense to refer to the introduction of DNA into a recipient host cell that changes the genotype and consequently results in a change in the recipient cell.

As used herein, “host cells” refers to cells that have been transformed with vectors constructed using recombinant DNA techniques and encoding at least one heterologous gene. In descriptions of processes for isolation of antibodies from recombinant hosts, the terms “cell” and “cell culture” are used interchangeably to denote the source of binding molecule unless it is clearly specified otherwise. In other words, recovery of polypeptide from the “cells” may mean either from spun down whole cells, or from the cell culture containing both the medium and the suspended cells.

E. coli is one prokaryotic host particularly useful for cloning the polynucleotides (e.g., DNA sequences) of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, are also useful for expression. Saccharomyces is a preferred yeast host, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian tissue cell culture may also be used to express and produce polypeptides encoding a binding molecule for use in the methods of the present invention (e.g., polynucleotides encoding binding molecules). See Winnacker, From Genes to Clones, VCH Publishers, N.Y., N.Y. (1987). Eukaryotic cells are actually preferred, because a number of suitable host cell lines capable of secreting heterologous proteins (e.g., intact binding molecules) have been developed in the art, and include CHO cell lines, various Cos cell lines, HeLa cells, myeloma cell lines, or transformed B-cells or hybridomas. Preferably, the cells are nonhuman. Exemplary host cell lines include, but are not limited to, DG44 and DUXB11 (Chinese Hamster Ovary lines, DHFR minus), BELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte) and 293 (human kidney). CHO cells are particularly preferred. In one embodiment, the cells are CHO cells. Host cell lines are typically available from commercial services, the American Tissue Culture Collection or from published literature and those skilled in the art are credited with ability to preferentially determine particular host cell lines which are best suited for the desired gene product to be expressed therein.

Expression vectors for mammalian cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

In one embodiment, a binding molecule for use in the methods of the invention is prepared by recombinant expression of immunoglobulin light and heavy chain genes in a host cell. To express a binding molecule recombinantly, a host cell is transfected with one or more recombinant expression vectors carrying DNA fragments encoding the immunoglobulin light and heavy chains of the binding molecule such that the light and heavy chains are expressed in the host cell and, preferably, secreted into the medium in which the host cells are cultured, from which medium a binding molecule can be recovered. Standard recombinant DNA methodologies are used to obtain antibody heavy and light chain genes, incorporate these genes into recombinant expression vectors, and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al., (eds.) Current Protocols in Molecular Biology, Greene Publishing Associates, (1989) and in U.S. Pat. No. 4,816,397 by Boss, et al.,

To express a binding molecule, DNAs encoding partial or full-length light and heavy chains may be inserted into expression vectors such that the genes are operatively linked to transcriptional and translational control sequences. In one embodiment, the expression vector and expression control sequences are chosen to be compatible with the expression host cell used. The binding molecule light chain gene and the binding molecule heavy chain gene may be inserted into separate vector or, more typically, both genes are inserted into the same expression vector. The binding molecule genes may be inserted into the expression vector by standard methods (e.g., ligation of complementary restriction sites on the binding molecule gene fragment and vector, or blunt end ligation if no restriction sites are present). Prior to insertion of the binding molecule light or heavy chain sequences, the expression vector may already carry binding molecule constant region sequences. For example, one approach to converting VH and VL sequences to full-length binding molecule genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the binding molecule chain from a host cell. The binding molecule chain gene can be cloned into the vector such that the signal peptide is linked in-frame to the amino terminus of the binding molecule chain gene. The signal peptide can be an immunoglobulin signal peptide or a heterologous signal peptide (i.e., a signal peptide from a non-immunoglobulin protein).

The vectors containing the polynucleotide sequences of interest (e.g. the binding molecule heavy and light chain encoding sequences and expression control sequences) can be transferred into the host cell by well-known methods, which vary depending on the type of cellular host.

When heavy and light chains are cloned on separate expression vectors, the vectors are co-transfected to obtain expression and assembly of intact immunoglobulins. Once expressed, the whole binding molecules, their dimers, individual light and heavy chains, or other immunoglobulin forms of the present invention can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure binding molecules of at least about 90 to 95% homogeneity are preferred, and 98 to 99% or more homogeneity most preferred, for pharmaceutical uses.

Host cells can also be used to produce portions of intact binding molecules, such as Fab fragments or scFv molecules. It will be understood that variations on the above procedures are within the scope of the present invention. For example, it may be desirable to transfect a host cell with DNA encoding either the light chain or the heavy chain (but not both) of a binding molecule of this invention. In addition, bifunctional binding molecules may be produced in which one heavy and one light chain are specific for a first antigen and the other heavy and light chain are specific for a second antigen of a different polypeptide by crosslinking the first binding molecule to a second binding molecule by standard chemical crosslinking methods.

When recombinant expression vectors encoding binding molecule genes are introduced into mammalian host cells, binding molecules are produced by culturing the host cells for a period of time sufficient to allow for expression of the binding molecule in the host cells or, more preferably, secretion of the binding molecule into the culture medium in which the host cells are grown. Binding molecules can be recovered from the culture medium using standard protein purification methods (discussed further below).

Host cells which expressing the desired binding molecule may be cultivated under a variety of cell culture conditions. For example, mammalian cells are typically grown in a media containing, for example, amino acids, salts (e.g., ferric nitrate nonahydrate, potassium chloride, magnesium sulfate, sodium chloride, and sodium dihydrogen phosphate), glucose, vitamins (e.g., ascorbic acid, folic acid, nicotinamide, riboflavin, B-12). Fetal bovine serum (FBS) may be included, but is optional. In one embodiment, a binding molecule preparation is obtained from cells cultured in serum-free medium. In another embodiment, binding molecule preparation is obtained from cells cultured in serum containing medium.

The choice of the host cell expression system dictates to a large extent the nature of the cell culture procedures to be employed. The selection of a particular mode of production, be it batch or continuous, spinner or air lift, liquid or immobilized can be made once the expression system has been selected. Accordingly, homogeneous suspension culture, e.g. in an airlift reactor, fluidized bed bioreactors, continuous stirrer reactor, immobilized or entrapped cell culture, e.g. hollow fiber bioreactors, microcapsules, on agarose microbeads or ceramic cartridges, roller bottle cultures, or stirred tank bioreactors, with or without cell microcarriers may variously be employed. The criteria for such selection are appreciated in the cell culture art.

Cell culture procedures for the large- or small-scale production of binding molecules are potentially useful within the context of the invention. Procedures including, but not limited to, a fluidized bed bioreactor, hollow fiber bioreactor, roller bottle culture, or stirred tank bioreactor system may be used, in the later two systems, with or without microcarriers, and operated alternatively in a batch, fed-batch, or continuous mode.

In one embodiment, a binding molecule is obtained from cells cultured in a stirred tank bioreactor system. In another embodiment, a binding molecule is obtained from cells cultured in a hollow fiber bioreactor. In one embodiment, a fed-batch culture procedure is employed. In another embodiment, a batch culture is employed. In fed-batch culture, the mammalian host cells and culture medium are supplied to a culturing vessel initially and additional culture nutrients are fed, continuously or in discrete increments, to the culture during culturing, with or without periodic cell and/or product harvest before termination of culture. The fed-batch culture can include, for example, a semi-continuous fed-batch culture, wherein periodically whole culture (including cells and medium) is removed and replaced by fresh medium. Fed-batch culture is distinguished from batch or simple-batch culture in which all components for cell culturing (including the cells and all culture nutrients) are supplied to the culturing vessel at the start of the culturing process. Fed-batch culture can be further distinguished from perfusion culturing insofar as the supernate is not removed from the culturing vessel during the process (in perfusion culturing, the cells are restrained in the culture by, e.g., filtration, encapsulation, anchoring to microcarriers, etc., and the culture medium is continuously or intermittently introduced and removed from the culturing vessel).

Further, the cells of the culture may be propagated according to any scheme or routine that may be suitable for the particular host cell and the particular production plan desired. Therefore, the invention contemplates a single-step or multiple-step culture procedure. In a single-step culture the host cells are inoculated into a culture environment and the processes of the instant invention are employed during a single production phase of the cell culture. Alternatively, a multi-stage culture is envisioned. In the multi-stage culture cells may be cultivated in a number of steps or phases. For instance, cells may be grown in a first step or growth phase culture wherein cells, possibly removed from storage, are inoculated into a medium suitable for promoting growth and high viability. The cells may be maintained in the growth phase for a suitable period of time by the addition of fresh medium to the host cell culture.

According to one aspect of the invention, fed-batch or continuous cell culture conditions are devised to enhance growth of the mammalian cells in the growth phase of the cell culture. In another aspect, a bulk cell culture method is devised for cell growth. During fed-batch, or continuous cell culture conditions, the growth phase cells are grown under conditions and for a period of time that is maximized for growth. Culture conditions, such as temperature, pH, dissolved oxygen (DO₂), and the like, are those used with the particular host and will be apparent to the ordinarily-skilled artisan. The pH is adjusted to an appropriate level generally between about 6.5 and 7.5 using either an acid (e.g., CO₂) or a base (e.g., Na₂CO₃ or NaOH). A suitable temperature range for culturing mammalian cells such as CHO cells is between about 30 to 40° C. and generally about 37° C. and a suitable DO₂ is between 5-90% of air saturation. However, higher or lower ranges for any of these conditions may be necessary depending on the cell type.

At a particular stage the cells may be used to inoculate a production phase or step of the cell culture. Alternatively, as described above the production phase or step may be continuous with the inoculation or growth phase or step.

According to the invention, the cell-culture environment during the production phase of the cell culture is controlled. In one aspect, the production phase of the cell culture process is preceded by a transition phase of the cell culture in which parameters for the production phase of the cell culture are engaged.

Binding molecule production in mammalian, e.g., CHO, cells may employ a semi-continuous process whereby cells are cultured in a “seed-train” for various periods of time and are periodically transferred to inoculum fermentors to initiate the cell-amplification process en route to larger scale production of the polypeptide of interest. Thus, cells used for binding molecule production are in culture for various periods of time up to a maximum predefined cell age. The parameters of the cell culture process, such as seed density, pH, DO₂ and temperature during culture, duration of the production culture, operating conditions of harvest, etc. are a function of the particular cell line and culture medium used, and can be determined empirically, without undue experimentation.

### IV. Purification Methods of the Invention

The present invention features methods for reducing protein A contamination in a binding molecule preparation, e.g., a therapeutic binding molecule preparation, comprising residual protein A, or fragments thereof. As described in the appended examples, the pretreatment of a charge-modified depth filter with a solution having a basic pH, i.e., greater than about 8, efficiently removes residual protein A from a binding molecule preparation. Accordingly, the present invention provides methods for reducing protein A contamination in a binding molecule preparation comprising residual protein A, or fragments thereof, from a binding molecule preparation, e.g., a therapeutic binding molecule preparation, the method comprising, contacting a charge-modified depth filter with a solution having a pH greater than about 8 to obtain a pre-treated charge-modified depth filter, contacting the pre-treated charge-modified depth filter with the binding molecule preparation, and collecting the binding molecule preparation that flows through the filter to thereby obtain a binding molecule preparation having reduced protein A contamination. In one embodiment, the charged-depth filter is an anion exchange filter.

Pretreatment of the charge-modified depth filter involves contacting the filter with a solution having a pH greater than about 8. In one embodiment, the solution having a pH greater than about 8 is sodium hydroxide. In one embodiment, the concentration of sodium hydroxide is greater than about 0.05 Molar. The step of contacting the charge-modified depth filter may involve flushing the sodium hydroxide at a constant flux through the filter (i.e., using a vacuum or compressed air), or it may involve holding the sodium hydroxide in the filter without flow for greater than about, e.g., 1, 2, 3, 4, 5, 6, 7, 8, 9, 10, 15, 20 minutes, and then flushing the sodium hydroxide solution through the filter. In one embodiment, the sodium hydroxide in the filter is held without flow for greater than 5 minutes. Following the step of contacting the charge-modified depth filter with a solution having a pH greater than about 8, the filter may be contacting with a binding molecule preparation, or alternatively, it may be rinsed with, for example sterile sodium phosphate, having a molarity of 20-200 mM, and/or sterile, de-ionized water prior to contacting with a binding molecule preparation.

It is understood that the methods of the invention may be practiced in combination with any other method(s) utilized in the art for the purification and/or polishing of a binding molecule preparation, e.g., a therapeutic binding molecule preparation. For example, the purification of a binding molecule preparation typically involves numerous steps for removal of, for example, media components, cells, cell debris, virus particles, etc., in combination with selective isolation of binding molecules themselves, i.e., protein A affinity chromatography. The order of the steps may vary based on, for example, culture conditions, the buffering environment, and the isoelectric point of the antibody, and thus, the methods of the invention may involve upstream purification step(s) and/or downstream purification step(s) (upstream or downstream of protein A chromatography) such as, for example, centrifugation, depth filtration, anion exchange chromatography, cation exchange chromatography, hydrophobic interaction chromatography, or combinations thereof. Alternatively, the methods of the invention may be the final step before, for example, therapeutic formulation of the binding molecule.

In one embodiment, a binding molecule preparation is partially purified prior to the practice of the methods of the invention. For example, as described below, binding molecules, e.g., therapeutic binding molecules, are often produced recombinantly in mammalian cell culture fbr expression of these proteins (Chadd and Chamow (1991) Curr. Opin. Biotechnol. 12:188). Typically, the binding molecules are secreted into the culture medium from which they are recovered, however, it is within the scope of the invention to recover the binding molecules from lysates of host cells as well. Accordingly, an optional step prior to the removal of residual protein A from a binding molecule preparation is one or more harvest and/or clarification steps aimed at separating the solid components of the cell culture process (i.e., mammalian cells and cell debris) from the liquid phase that contains the binding molecule. A clarification step avoids the concentration of particulate material in subsequent steps which often include chromatography, and additionally prevents the particulate contaminants from dissolving during the later steps of the purification process and contaminating the purified target substance. Both centrifugation (Maybury, et al. (2000) Biotechnol. Bioeng. 67:265) and cross-flow microfiltration (Parnham and Davis (1995) Biotechnol. Bioeng. 47:155) are frequently used for cell culture harvest. Even the most effective centrifuges may not be able to remove all cellular components and thus filtration steps that can remove cell debris based on size may optionally follow centrifugation. For example, depth filters may be employed to remove larger, insoluble contaminants that would otherwise clog downstream operations (e.g., chromatography columns, filters, filtration membranes, and the like). Additional, non-limiting methods for clarification are well-known in the purification art, and include, for example, gravity separation, precipitation, flocculation-assisted sedimentation, decanting, normal filtration, sieving, absorption, adsorption, and tangential flow filtration.

Following a clarification step(s), a binding molecule preparation may be purified by affinity chromatography, e.g., protein A affinity chromatography, to specifically isolate binding molecules. Alternatively or optionally, the binding molecule preparation may be purified by other methods, e.g., anion exchange chromatography, cation exchange chromatography, size exclusion chromatography, fractionation, and the like using commercially available columns and filters. One of skill in the art can readily determine the purification steps that are necessary and the order of such steps prior to affinity chromatography, e.g., protein A affinity chromatography. Following an affinity chromatography, e.g., protein A affinity chromatography, purification step, the methods of the invention are used to reduce residual protein A contamination in the binding molecule preparation. Additional purification steps, such as those described herein, may be used to further purify the binding molecule preparation, for example, prior to, pharmaceutical formulation, including, for example, tangential flow filtration, and/or steps to and/or remove viruses and retroviruses that might potentially be present in the cell culture fluid of continuous mammalian cell lines. A significant number of viral clearance steps are known to one of skill in the art including, for example, additional filtration steps, treatment with chaotropes such as urea or guanidine, pH extremes, detergents, heat, chemical derivatization, such as formaldehyde, proteases, conventional separation, such as ion-exchange or size exclusion chromatography, organic solvents, etc.

The amount of protein A contamination in a binding molecule preparation may be measured at any point(s) following protein A affinity chromatography. Methods to measure the amount of residual protein A contamination in a binding molecule preparation are known in the art and are include, for example, immunoenzymatic assays commercially available from, for example Cygnus Technologies, Inc. or Repligen Corporation. The methods of the invention may be used to remove residual protein A from a binding molecule preparation in which the initial residual protein A concentration is greater than about, for example, 25, 30, 35, 40, 45, 50, 55, 60, 65, 70, 75, 80, 85, 90, 95, 100 ppm (parts per million). In one embodiment, the initial residual protein A concentration is greater than about 30 ppm. The methods of the invention may reduce the concentration of residual protein A contamination in a binding molecule preparation following protein A affinity chromatography to about 35, 30, 25, 20, 15, 10, 5, 4.5, 4, 3.5, 3, 2.5, 2, 1.5, 1, 0.5 ppm. In one embodiment, the concentration of residual Protein A is reduced to less than about 20 ppm. In another embodiment, the concentration of residual Protein A is reduced to less than about 10 ppm. In yet another embodiment, the concentration of residual Protein A is reduced to less than about 5 ppm. In one embodiment, the concentration of residual Protein A is reduced to less than about 2.5 ppm. In another embodiment, the concentration of residual Protein A is reduced to less than about 1 ppm.

Clarification and/or chromatography steps are optional and may not be necessary. Furthermore, one or a combination of purification steps described herein can be used to purify a binding molecule preparation. In one embodiment, a binding molecule preparation is clarified, e.g., to remove cells and cell debris prior to affinity chromatography, e.g., protein A affinity chromatography.

In addition to the purification steps described above, incubation and/or washing steps may be required after each step or combination of steps in the purification process. Incubation steps can vary from about 5 seconds to several hours, preferably from about 5 minutes to about 24 hours. However, the incubation time will depend upon the assay format, type of sample, volume of solution, concentrations, and the like. Usually the assays will be carried out at ambient temperature, although they can be conducted over a range of temperatures, such as 10° C. to 40° C.

### V. Uses of the Purified Binding Molecules

Many uses for binding molecule preparations purified according to the disclosed methods are provided, including diagnostic and therapeutic uses.

The binding molecules described herein can be used in immunoassays, such as enzyme immunoassays. Binding molecules can also be used for in vitro or in vivo immunodiagnosis of various diseases such as autoimmune diseases and disorders, infectious disease and disorders, and cancer. To facilitate this diagnostic use, an antibody which binds to, e.g., a tumor associated antigen can be conjugated with a detectable marker (e.g., a chelator which binds a radionuclide). Other non-therapeutic, diagnostic uses for the antibody will be apparent to the skilled practitioner.

For diagnostic applications, the binding molecule typically will be labeled directly or indirectly with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I; a fluorescent or chemiluminescent compound, such as fluorescein isothiocyanate, rhodamine, or luciferin; or an enzyme, such as alkaline phosphatase, beta-galactosidase or horseradish peroxidase. Any method known in the art for separately conjugating the binding molecule to the detectable moiety may be employed, including those methods described by Hunter et al., Nature 144:945 (1962); David et al., Biochemistry 13:1014 (1974); Pain et al., J. Immunol. Meth. 40:219 (1981); and Nygren, J. Histochem. and Cytochem. 30:407 (1982).

The binding molecules of the present invention may be employed in any known assay method, such as competitive binding assays, direct and indirect sandwich assays, and immunoprecipitation assays. Zola, Monoclonal Antibodies: A Manual of Techniques, pp. 147 158 (CRC Press, Inc., 1987). Competitive binding assays rely on the ability of a labeled standard to compete with the test sample analyte for binding with a limited amount of binding molecule. The amount of analyte in the test sample is inversely proportional to the amount of standard that becomes bound to the binding molecule. To facilitate determining the amount of standard that becomes bound, the binding molecule is generally insolubilized before or after the competition, so that the standard and analyte that are bound to the binding molecule may conveniently be separated from the standard and analyte which remain unbound.

The binding molecules also are useful for the affinity purification of an antigen of interest from recombinant cell culture or natural sources.

Therapeutic uses for the binding molecules purified using the method described herein are also provided. In one embodiment, a binding molecule prepared as described herein is incorporated in a pharmaceutical formulation. In one embodiment, a binding molecule prepared as described herein and incorporated in a pharmaceutical formulation is used to treat an autoimmune disease or disorder. Non-limiting examples of autoimmune diseases and disorders having an autoimmune component that may be treated according to the invention include type I diabetes, arthritis (including rheumatoid arthritis, juvenile rheumatoid arthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia greata, allergic responses due to arthropod bite reactions, Crohn's disease, iritis, conjunctivitis, keratoconjunctivitis, ulcerative colitis, asthma, allergic asthma, cutaneous lupus erythematosus, scleroderma, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis. In preferred embodiments, the disease is selected from the group consisting of type I diabetes, multiple sclerosis and rheumatoid arthritis.

Therapeutic formulations of the binding molecule are prepared for storage by mixing the antibody having the desired degree of purity with optional physiologically acceptable carriers, excipients, or stabilizers, as described below.

An effective amount of antibody to be employed therapeutically will depend, for 5 example, upon the therapeutic objectives, the route of administration, and the condition of the patient. Accordingly, it may be necessary for the skilled practitioner to titer the dosage and modify the route of administration as required to obtain the optimal therapeutic effect. A typical daily dosage might range from about 1 mg/kg to up to 10 mg/kg or more, depending on the factors mentioned above. Additionally dosages independent of body weight may range from, for example, 0.1 mg-10 mg per subject over a single course of therapy or a regiment that may include multiple doses over time. Typically, the clinician will administer antibody until a dosage is reached that achieves the desired effect.

### VI. Pharmaceutical Compositions

The purified binding molecules prepared according to the methods of the present invention can be incorporated into pharmaceutical compositions suitable for administration to a subject. Typically, the pharmaceutical composition comprises a binding molecule and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is provided. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, a binding molecule is prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds ties preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The pharmaceutical compositions can be included in a container, pack, or dispenser together with instructions for administration.

This invention is further illustrated by the following examples, which should not be construed as limiting. The contents of all references, patents and published patent applications cited throughout this application, as well as the Figures and Tables, are incorporated herein by reference.

### EXAMPLES

### Example 1

### Methods for Removal of Residual Protein A

### A. Anti-CD3 Purification Process Overview

The anti-CD3 purification process utilizes affinity purification as an initial purification step. The affinity resin that is used is POROS A50 and is manufactured by Applied Biosystems. The affinity purification uses low pH elution followed by immediate neutralization to a pH>5.0. In line with all conventional therapeutic manufacturing processes, the anti-CD3 process incorporates, several other polishing steps for removal of low levels of contaminating residuals such as residual CHO DNA (Chinese Hamster Ovary Cells or media components. The process also includes two ontological viral inactivation and/or clearance steps for removal of adventitious viruses.

The isoelectric point (pI) of an antibody dictates the order of the polishing steps in the process. It is important that at least one be a binding step where the antibody binds to a matrix based on charge or hydrophobicity. The pI of anti-CD3 is 8.3 and therefore the first polishing step is a cation exchange (SP-Sepharose Fast Flow) binding step. anti-CD3 binds to the SP-Sepharose under buffer condtions that impart a net positive charge on the antibody. Since the net charge on the SP-Sepharose resin is negative, the antibody binds to the resin and can be eluted with buffers of increasing pH and ionic strength.

Next, the antibody is passed over an anion exchanger that is either in a filter format (Cuno VR06) or a column chromatography format (Q-Sepharose Fast Flow). The net positive charge of both the antibody and anion exchange resin ensures that the antibody flows through whereas any negatively charged contaminants (such as residual CHO DNA or viruses) will bind and therefore be removed from the antibody product stream.

### B. Removal of Residual Protein A by Cuno VR06 Depth Filtration

The optimization of the purification process for the anti-CD3 led to investigation of residual protein A in the process streams. Following the affinity purification step, the levels were greater than or equal to 60 ppm and therefore, the goal was to decrease these levels in the subsequent downstream purification steps. Efforts were made to reduce the levels of residual protein A to below 20 ppm by conventional methods such as by (1) adding chemical agents to chelate the activity of the contaminating proteases (namely EDTA for serine proteases) (2) optimizing the SP Sepharose wash and elution conditions to exploit the biochemical differences between free antibody and antibody complexed with protein A and (3) decreasing the operational temperature of the affinity step to slow that catalytic activity of any proteases. All of these methods aided in decreasing the levels of residual protein A, but only by approximately 50%, i.e., to about 30 ppm.

The step that was found was responsible for removal of the residual protein A was the Cuno VR06 filtration step. It was found, that the residual protein A levels were decreased only upon pre-treatment of the filter with 0.1 N Sodium Hydroxide. More specifically, the filters were flushed with 0.1 N Sodium Hydroxide, held for 20 minutes and then flushed dry. Next, the filter was equilibrated with neural buffer and labeled ready for use.

In contrast, when following the manufactures recommended protocol of flushing the cartridge with water for injection (WFI) and to use (with or without autoclaving) only, the filters were not effective at removing the residual protein A. Therefore, it was found that pre-treatment of the Cuno VR06 filter with Sodium Hydroxide was critical and extensive investigation has led to the findings listed below in Tables 1 and 2.

TABLE 1

| | Residual Protein |
|---|---|
| Cuno VR06* Pre-Treatment (10-12° C. or 18-22° C.) | Removed (≧20 ppm) |
| 150 ml H₂0 Flush Only | No |
| 150 ml H₂0 Flush with Autoclave Cycle | No |
| 150 ml H₂0 Flush/≧7 ml 0.1 N NaOH Flush^{#}/≧7 ml Sodium Phosphate Flush | Yes |
| ≧7 ml 0.1 N NaOH Flush Only | Yes |
| ≧7 ml 0.1 N NaOH Flush/≧7 ml Sodium Phosphate Flush | Yes |
| 150 ml H₂0 Flush/≧7 ml 0.1 N NaOH Flush/≧7 ml Sodium Acetate Flush | Yes |
| ≧7 ml 0.1 N NaOH Flush/≧7 ml Sodium Acetate Flush | Yes |

^{#}NaOH can be passed through under pressure, under flow or held static for 20 min
*Data based on 24-cm² surface area Cuno VR06 filters where one hold up volume is approximately 7 ml

TABLE 2

| | Concentration | Residual ProA | Residual ProA |
|---|---|---|---|
| Sample ID | (mg/ml) | (ng/ml) | (ppm) |
| SP - Sepharose Load | 0.87 | 48.5 | 55.7 |
| Cuno Load (SP- | 9.50 | 461 | 48.5 |
| Eluate) | | | |
| Cuno Flow Through | 5.98 | 3.99 | 0.7 |

Without wishing to be bound by theory, it is thought that the treatment of the filter with sodium hydroxide may change the charge characteristics on the membrane or the diatomaceous Earth enough causing the antibody-residual protein A complex to bind to the membrane while allowing the antibody to flow through. The difference in the charge and hydrophobicity of the antibody-residual protein A complex as compared to the charge of the antibody alone may be sufficient enough so that this binding event occurs. The isoelectric point of the antibody is approximately 8.0 whereas the isoelectric point of free protein A is approximately 5.0. Again, a complex of both may shift the pI of the complex enough so that binding occurs.

Alternatively, the treatment of the membrane with sodium hydroxide may relax the compressed structure of the filter membrane enough so that more charged groups are exposed to the product stream or that more surface area is available for the second mechanism of removal, physical removal. The slight difference in molecular size of the antibody-residual protein A complex may be sufficient enough so that the increased surface area of the filter after sodium hydroxide may allow the filter to act as a molecular sieve, essentially filtering out the contamination.

### Example 3

### Larger Scale Functionality of the Cuno VR06 Depth Filter for the Removal of Residual Protein A

Afinnity capture chromatography utilizing Poros A50 protein A resin was used to purify the anti-CD3 antibody from the cell culture harvest. The Poros A chromatography resin used to capture the anti-CD3 antibody was a recombinant protein A bound to a Poros A50 resin manufactured by Applied Biosystems, Bedford, Mass. The affinity purification was accomplished using a 40 liter column under standard operating conditions which included binding, wash and low pH elution steps. Following elution the antibody was neutralized and diluted in preparation for the subsequent cation exchange chromatography step. Samples of the Poros A eluate were taken in order to assess the levels of residual protein A and other purification process contaminants. Next, the anti-CD3 antibody was bound to a 55 liter cation exchange chromatography column (SP—Sepharose FF) under acetic, low salt conditions. Following the binding step, the column was washed under similar buffering conditions that were optimized to prevent elution of the antibody or irreversible binding under moderate buffering salt concentrations. Elution from the cation exchange resin was achieved using a basic buffer solution with an increase concentration of buffering salts. Samples of the cation exchange eluate were also taken to assess the levels of residual protein A and other purification process intermediates.

The sanitization procedure of the Cuno VR06 depth filter was found to be necessary for effective removal of residual protein A at small scale (24 cm²). The pre-treatment at GMP scale was also preformed using this method. Briefly, the Cuno VR06 filters (3 m²) were flushed with 150 liters of water for injection (WFI) and then sanitized with 70 liters of 0.1 M sodium hydroxide, pH 12.0. The sodium hydroxide was held in the filters for 20 minutes at 10-14 C. Following the hold, the filter was neutralized by flushing with 50 liters of 100 mM Sodium Phosphate, pH 7.4. After neutralization the cation exchange elute was flushed through the filters using 10 psig compressed air. Finally, an additional three filter hold-up volumes were flushed through the filters to achieve acceptable product recovery. After filtration, samples were taken to assess the levels of residual protein A and other purification process intermediates.

Poros A50, cation exchange and Cuno VR06 purification process intermediates were assessed for residual protein A levels using the Cygnus Protein A ELISA kit (catalog #F050H). Poros A50 and cation exchange intermediates were diluted 1:50 and 1:100 in duplicate and the Cuno VR06 filter intermediate was assessed undiluted, 1:2, 1:20 and 1:50, each dilution in duplicate. There was not any evidence of buffer interference in the assay as judged by the results from the above mentioned samples as reported in Table 3, or the spike and blank controls that were run in the assay.

The residual protein A level seen in the Poros A eluate at this scale were similar to what was seen at smaller scale. At the 40 L column scale the residual protein A levels in the Poros A eluate ranged from 67-70 ppm (Table 3) for the dilutions tested. At smaller scales (4-2000 ml) the levels observed were slightly lower at approximately 55 ppm (Table 2). In addition, the residual protein A levels observed in the cation exchange eluate (SP-Sepharose) at larger GMP scale were almost identical at 48-56 ppm (Table 3) to what was seen in the smaller lab and scale-up lab purifications (48 ppm in Table 2) that were assessed.

The pretreatment of the Cuno VR06 depth filter was found to be necessary in small lab scale experiments as well as larger scale purification campaigns. In the smaller scale experiments, the levels of residual protein A after the Cuno filtration step were typically found to be less than 10 ppm (Table 2). At the larger scale observed levels of residual protein A levels ranged from 9.2-11.4 ppm (Table 3). Although the contaminating protein A or protein A—Antibody complex was not removed completely by this method, it was effective at reducing the levels by approximately 4 fold to below 20 ppm in the final drug substance.

TABLE 3
Residual protein A levels for affinity chromatography (Poros
A50), cation exchange chromatography (SP-Sepharose FF) and Cuno
YR06 depth filtration intermediates were assessed using the Cygnus
Protein A ELISA Assay (catalog #F050H).

| | | Average [Protein A] | |
|---|---|---|---|
| Test Article | DF | ng/ml | Average ppm |
| Affinity Column Eluate | 50 | 0.699 | 70 |
| Affinity Column Eluate | 100 | 0.333 | 67 |
| Cation Exchange Eluate | 50 | 10.609 | 48.2 |
| Cation Exchange Eluate | 100 | 6.223 | 56.6 |
| Cuno VR06 Flow Through | 1 | 26.255 | 10.9 |
| Cuno VR06 Flow Through | 2 | 13.678 | 11.4 |
| Cuno VR06 Flow Through | 20 | 1.179 | 9.8 |
| Cuno VR06 Flow Through | 50 | 0.444 | 9.2 |

Each sample dilution was run in duplicate the average concentration of protein A in ng/ml are reported in the table. The ppm calculation was based on the protein concentration of the sample as measured by absorbance at 280 nm.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for reducing protein A contamination in a binding molecule preparation comprising residual protein A, the method comprising,
contacting a charge-modified depth filter with a solution having a pH greater than about 8 to obtain a pre-treated charge-modified depth filter;
contacting the pre-treated charge-modified depth filter with the binding molecule preparation; and
collecting the binding molecule preparation that flows through the filter to thereby obtain a binding molecule preparation having reduced protein A contamination.

2. The method of claim 1, wherein the charged-depth filter is an anion exchange filter.

3. The method of claim 1, wherein prior to reducing protein A contamination the binding molecule preparation is purified by a process selected from the group consisting of: centrifugation, depth filtration, protein A affinity chromatography, anion exchange chromatography, cation exchange chromatography, or a combination thereof.

4. The method of claim 1, further comprising purifying the binding molecule preparation by a process selected from the group consisting of: anion exchange chromatography, cation exchange chromatography, filtration, or a combination thereof.

5. The method of claim 1, wherein the charge-modified depth filter comprises a matrix selected from the group consisting of: paper, glass fibers, nylon, polyolefin, carbon, ceramics, diatomaceous Earth and cellulose.

6. The method of claim 5, wherein the matrix is cellulose.

7. The method of claim 1, wherein the solution having a pH greater than about 8 is sodium hydroxide.

8. The method of claim 6, wherein the concentration of the sodium hydroxide solution is greater than about 0.05 M NaOH

9. The method of claim 6, wherein the sodium hydroxide is either flushed at a constant flux through the filter or held without flow for greater than 5 minutes and then flushed through the filter.

10. The method of claim 1, further comprising the step of contacting the pre-treated filter with sodium phosphate or water prior to contact with the antibody preparation.

11. The method of claim 1, wherein the initial concentration of residual Protein A is greater than 30 ppm.

12. The method of claim 1, wherein the concentration of residual Protein A is reduced to less than about 20 ppm.

13. The method of claim 1, wherein the concentration of residual Protein A is reduced to less than about 10 ppm.

14. The method of claim 1, wherein the concentration of residual Protein A is reduced to less than about 5 ppm.

15. The method of claim 1, wherein the concentration of residual Protein A is reduced to less than about 2.5 ppm.

16. The method of claim 1, wherein the concentration of residual Protein A is reduced to less than about 1 ppm.

17. The method of claim 1, wherein the binding molecule preparation comprises a monoclonal binding molecule.

18. The method of claim 1, wherein the binding molecule preparation comprises a chimeric binding molecule.

19. The method of claim 1, wherein the binding molecule preparation comprises a humanized binding molecule.

20. The method of claim 1, wherein the binding molecule preparation comprises a an IgG1 constant region.

21. The method of claim 1, wherein the binding molecule preparation is obtained from cells cultured in serum-free or serum containing medium.

22. The method of claim 1, wherein the binding molecule is obtained from cells cultured in a hollow fiber or stirred tank bioreactor in batch or fed batch configuration

23. The method of claim 1, further comprising incorporating the binding molecule preparation into a pharmaceutical formulation.
